(19) United States
(12) Patent Application Publication
(10) Pub. No.: US 2024/0055140 A1
(43) Pub. Date: Feb. 15, 2024

(54) SYSTEMS AND METHODS FOR RECOMMENDING MEDICALLY-RELEVANT ARTICLES

(71) Applicant: Optum, Inc., Minnetonka, MN (US)

(72) Inventors: Gregory J. BOSS, Saginaw, MI (US); Adam RUSSELL, Arlington, MA (US); Ramprasad Anandam GADDAM, Mumbai (IN); Robert Kahn ROSSMILLER, Brookfield, WI (US)

(73) Assignee: Optum, Inc., Minnetonka, MN (US)

(21) Appl. No.: 17/885,975

(22) Filed: Aug. 11, 2022

(51) Int. Cl. G16H 70/00; G16H 10/60; G16H 40/20

(52) U.S. Cl. CPC G16H 70/00; G16H 10/60; G16H 40/20

(57) ABSTRACT

Methods, systems, and non-transitory computer readable mediums are disclosed for recommending medically-relevant articles. One method comprises receiving health data associated with a patient, storing the health data in a data structure as historical patient data, identifying new appointment scheduling data associated with the patient, determining an appointment subject associated with the new appointment scheduling data, determining a correlation between the new appointment scheduling data and the historical patient data, searching one or more databases for one or more relevant articles, retrieving the one or more relevant articles, generating a recommendation for the one or more relevant articles, and presenting the recommendation for the one or more relevant articles to a user through at least one of a news feed, a calendar, or an electronic medical record (EMR) associated with the patient.

### TECHNICAL FIELD

Various embodiments of the present disclosure relate generally to systems and methods for recommending relevant articles, and more particularly to, systems, computer-implemented methods, and non-transitory computer readable mediums for recommending medically-relevant articles based on data associated with a new appointment for a patient and historical patient data stored in a data structure.

### BACKGROUND

Clinicians often rely on understanding a patient's current medical condition and past medical history to make informed, appropriate decisions about providing optimal care. The total amount of patients a clinician sees per day and the nature of each patient visit may limit the amount of time a clinician is able to devote to preparing for each patient's appointment. In some cases, a clinician may need to treat a patient experiencing health issues that require additional background information and/or research. Further, since therapies, treatments, and diagnoses may change with time, clinicians are in need of the latest information relevant to their patients. As such, medical literature serves as an important resource in clinical decision making.

Existing methods for accessing medical literature that is relevant to a patient include searching medical databases, which requires a clinician to perform a manual search, often by inputting keywords into a search engine, in order to find relevant literature (e.g., articles). However, such methods are considered to be time intensive, since they may require a clinician to spend a significant amount of time searching one or more databases, filtering through the results, and then reviewing the relevant information prior to each patient visit. Clinicians may also be tasked with taking additional time out of their schedule to recall and/or retrieve specific details from the entire pool of patients currently under their care. In addition, the relevancy of the articles recommended through existing systems and methods is largely dependent on a clinician's search skills (e.g., search term/keyword choices). Conventional methods of obtaining relevant information, which require manual searching, also limit the amount of concepts that a clinician may search at one time.

Therefore, a need exists for a system and method for automatically recommending relevant articles to clinicians for each patient visit based on data associated with a new appointment scheduled for a patient and historic patient data, and providing the relevant articles in an easily digestible format that saves the clinicians' time and fosters well-informed clinical decisions.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, computer-implemented methods, systems, and non-transitory computer readable mediums are disclosed for identifying new appointment scheduling data for a patient, determining an appointment subject, determining (e.g., by using a data structure) a correlation between the new appointment scheduling data and historical patient data, searching one or more databases for one or more relevant articles, and generating a recommendation for the one or more relevant articles. The computer-implemented methods, systems, and non-transitory computer readable mediums of the present disclosure may also present the one or more relevant articles to a user through a news feed, a calendar, and/or a patient electronic medical record.

In one aspect, an exemplary computer-implemented method may include: receiving, by a processor, health data associated with a patient; storing, by the processor, the health data associated with the patient in a data structure as historical patient data; identifying, by the processor, new appointment scheduling data associated with the patient; determining, by the processor, an appointment subject associated with the new appointment scheduling data based on the new appointment scheduling data; determining, by the processor and using the data structure, a correlation between the new appointment scheduling data and the historical patient data based on the new appointment scheduling data and the historical patient data; searching, by the processor, one or more databases for one or more relevant articles using query data related to at least the appointment subject; retrieving, by the processor, the one or more relevant articles from the one or more databases; and generating, by the processor, a recommendation for the one or more relevant articles. The method may further include: presenting the recommendation for the one or more relevant articles to a user on a user device through at least one of a news feed, a calendar, or an electronic medical record (EMR) associated with the patient.

In another aspect, an exemplary system may include a memory having processor-readable instructions stored therein and a processor configured to access the memory and execute the processor-readable instructions to perform operations. The operations may include: receiving health data associated with a patient; storing the health data associated with the patient in a data structure as historical patient data; identifying new appointment scheduling data associated with the patient; determining an appointment subject associated with the new appointment scheduling data based on the new appointment scheduling data; determining, by using the data structure, a correlation between the new appointment scheduling data and the historical patient data based on the new appointment scheduling data and the historical patient data; searching one or more databases for one or more relevant articles using query data related to at least the appointment subject; retrieving the one or more relevant articles from the one or more databases; and generating a recommendation for the one or more relevant articles. The operations may further include presenting the recommendation for the one or more relevant articles to a user through at least one of a news feed, a calendar, or an electronic medical record (EMR) associated with the patient.

In yet another aspect, disclosed is a non-transitory computer-readable medium storing a set of instructions that, when executed by a processor, may perform operations for recommending medically-relevant articles, wherein the operations include: receiving health data associated with a patient; storing the health data associated with the patient in a data structure as historical patient data; identifying new appointment scheduling data associated with the patient; determining an appointment subject associated with the new appointment scheduling data based on the new appointment scheduling data; determining, by using the data structure, a correlation between the new appointment scheduling data and the historical patient data based on the new appointment scheduling data and the historical patient data; searching one or more databases for one or more relevant articles using query data related to at least the appointment subject; retrieving the one or more relevant articles from the one or more databases; and generating a recommendation for the one or more relevant articles. The operations may further include presenting the recommendation for the one or more relevant articles to a user through at least one of a news feed, a calendar, or an electronic medical record (EMR) associated with the patient.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 depicts a block diagram of an exemplary system for recommending relevant articles to clinicians, according to one or more embodiments.

FIG. 2 depicts a flowchart illustrating an exemplary method for recommending relevant articles to clinicians, according to one or more embodiments.

FIG. 3 depicts a flowchart illustrating an exemplary method for recommending relevant articles based on article read time and time slots in a schedule, according to one or more embodiments.

FIG. 4 depicts an exemplary graph data structure representing multiple patient visits, according to one or more embodiments.

FIG. 5 depicts an exemplary schema of data in a graph data structure for a single patient, according to one or more embodiments.

FIG. 6 depicts an exemplary schema of data in a graph data structure for multiple appointments, according to one or more embodiments.

FIG. 7 depicts an exemplary graphical user interface (GUI) displaying a clinician calendar, according to one or more embodiments.

FIG. 8 depicts another exemplary GUI displaying a clinician calendar, according to one or more embodiments.

FIG. 9 depicts an exemplary GUI displaying an electronic medical record (EMR), according to one or more embodiments.

FIG. 10 depicts exemplary personalized feed displays, according to one or more embodiments.

FIG. 11 illustrates an implementation of a computer system that may execute techniques presented herein.

### DETAILED DESCRIPTION

Various embodiments of the present disclosure relate generally to providing recommendations. More particularly, various embodiments of the present disclosure relate to systems, computer-implemented methods, and non-transitory computer readable mediums for recommending medically-relevant articles based on data associated with a new appointment for a patient and historical patient data stored in a data structure.

As discussed above, clinicians may consult medical articles when preparing for patient appointments. Traditionally, clinicians are tasked with conducting their own searches to obtain medically-relevant articles. However, a significant amount of time may be expended conducting an effective search and the search may still be limited by a number of factors, including only being able to search one concept at a time and finding articles that may not be the most relevant. Clinicians may also fail to recognize certain relationships between a current patient condition and a past occurrence. In some instances, even when a clinician finds relevant materials, they may not have enough time to digest the information for each appointment while handling a full schedule of patients.

Therefore, the embodiments of the present disclosure are directed to solving, mitigating, or rectifying the above-mentioned issues by recommending one or more relevant articles for a patient's upcoming appointment and presenting the one or more relevant articles to a clinician through at least one of a news feed, a calendar, or an electronic medical record (EMR) associated with the patient in various levels of summarization. The systems and methods of the present disclosure may address the above-mentioned issues by receiving health data associated with a patient (e.g., EMRs, appointment records), storing the health data in a data structure (e.g., graph data structure) as historical patient data, identifying new appointment scheduling data associated with the patient, and determining an appointment subject associated with the new appointment scheduling data using, e.g., national language processing. The data structure may be used to determine a correlation between the new appointment scheduling data and the historical patient data. The data structure may be a weighted graph comprising a plurality of nodes representing each appointment of the patient, wherein each node is associated with an appointment subject, historical patient data, and one or more relevant articles, wherein the nodes are connected based on the respective appointment subject, and wherein edges between the connected nodes represent similarities between the connected nodes. One or more databases may be searched for the one or more relevant articles using query data related to at least the appointment subject. The one or more relevant articles may be retrieved from the one or more databases and a recommendation may be generated for the one or more relevant articles. The recommendation may be presented to the user in a convenient, user-friendly manner. The presentation of the recommended articles may also be customizable. The determined appointment subject of the new appointment scheduling data, data related to the correlation between the new appointment scheduling data and the historical patient data, and data related to the one or more relevant articles may also get stored in the data structure.

In addition, searching for one or more relevant articles may include computing a relevance score for each of the plurality of articles stored in the one or more databases, wherein the relevance score is relative to the appointment subject and/or the correlation between the new appointment scheduling data and the historical patient data. The present disclosure also encompasses querying the one or more databases with query data generated based on key phrases extracted from one or more articles associated with related historical patient data or by combining key phrases extracted from the new appointment scheduling data, related historical patient data, and one or more articles associated with the related historical patient data. Further, generating the recommendation for the one or more relevant articles may also include determining one or more time slots in a schedule of a user (e.g., clinician), determining a duration of each of the one or more time slots, determining a read time for each of the one or more relevant articles and recommending the one or more relevant articles based on the respective one or more read times and the duration of each of the one or more time slots.

The present disclosure provides systems, methods, and non-transitory computer readable mediums configured to receive health data associated with a patient, store the health data in a data structure as historical patient data, identify new appointment scheduling data associated with the patient, determine an appointment subject associated with the new appointment scheduling data, determine a correlation between the new appointment scheduling data and the historical patient data, search one or more databases for one or more relevant articles, retrieve the one or more relevant articles, and generate a recommendation for the one or more relevant articles. The following embodiments describe systems, computer-implemented methods, and non-transitory computer readable mediums for recommending medically-relevant articles.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The term “clinician” may include, for example, without limitation, any person, organization, and/or collection of persons that provides medical care (i.e. health care provider). For example, a clinician may include a physician, a nurse, a psychologist, an optometrist, a veterinarian, a physiotherapist, a dentist, and a physician assistant.

As used herein, “articles” may include, for example, any publications or content related to research (e.g., medical research). Examples of articles include, but are not limited to, abstracts, digests, journals, news articles, research papers, e-books, web pages including blogs, guidelines, podcasts, periodicals, videos, and reports.

As used herein, “health data” may include, for example, any data associated with a patient and/or a patient appointment. Examples of health data include, but are not limited to, electronic medical records (EMRs), appointment records, medication records, clinician's notes and/or recordings, other point-of-care documentation, and discharge documentation.

As used herein, “historical patient data” may include, for example, any stored (e.g., archived) data associated with a patient and/or a past patient appointment. Examples of historical patient data include, but are not limited to, electronic medical records (EMRs), appointment scheduling data (e.g., appointment records), past medical procedure, previous diagnosis, and clinician's notes and/or recordings. Historical patient data may also include appointment subject(s) and articles (e.g., relevant articles) associated with a previous patient appointment.

As used herein, a “data structure” generally encompasses a structure, such as a table, matrix, or a graph, containing data associated with one or more patients and one or more patient visits in an organized manner. A “data structure” may also include and be referred to herein as a “graph data structure.” The graph data structure may include a graph (e.g., weighted graph or knowledge graph) made up of nodes (e.g., vertices) representing patient appointments each associated with patient data nodes and edges (e.g., links) connecting the nodes. Similarity between connected nodes may be represented by the weight of the edges (e.g., edge weight).

The term “appointment” may include, for example, without limitation, any visit or meeting that a patient has with a clinician.

The term “new appointment scheduling data” may include, for example, without limitation, any information associated with the scheduling of a new patient appointment. For example, new appointment scheduling data may include a new appointment record (e.g., date and time of scheduled appointment), a reason for the patient visit, symptoms that the patient is experiencing, patient identification information (e.g., name and contact information), clinician/health care provider information (e.g., name), and administrative details (e.g., insurance and payment information).

The term “appointment subject” refers to a subject or context of a patient appointment. Categories for the appointment subject may include, but are not limited to, one or more of a side effect of a medication, a new patient concern (e.g., new injury or new symptom), an allergy, a progression of a health condition, genetic predisposition, a post-operative follow-up or a post-operative complication.

The term “query data” may include, for example, without limitation, any data used to search or query one or more databases. The query data may include, but is not limited to, keywords, key phrases, and codes/terms (e.g., from International Classification of Diseases (ICD)-9, ICD-10, and Systemized Nomenclature of Medicine-Clinical Terms (SNOMED CT)).

The terms “electronic medical records (EMRs)” and “electronic health records (EHRs)” refer to electronic records comprising patient medical history information. For example, EMRs may include, but are not limited to, patient demographic information (e.g., name address, phone number, gender, date of birth, etc.) and medical/health history information (e.g., symptoms, diagnoses, allergies, conditions, medications, lab/test results, and referral information etc.).

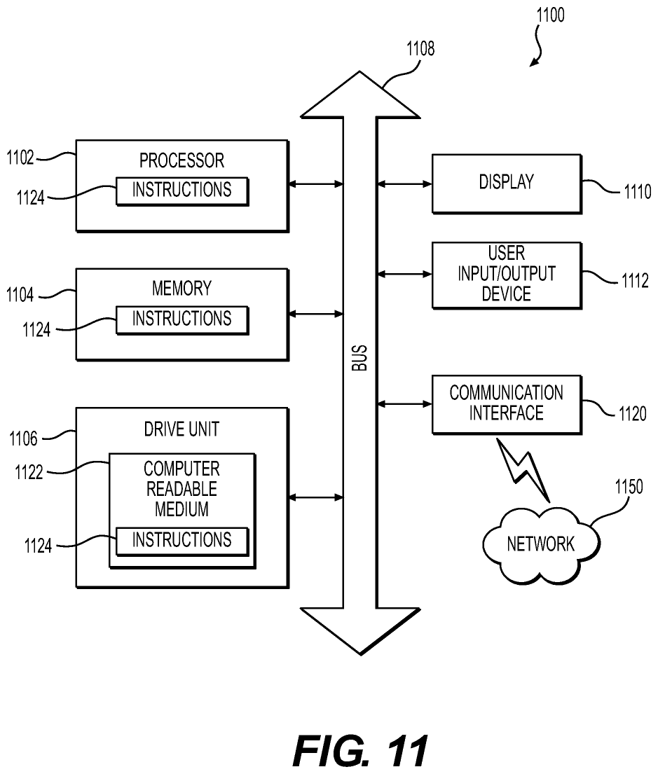

The terms “device” and “user device” may include, for example, without limitation, any electronic equipment, controlled by a processor (e.g., central processing unit (CPU)), for inputting information or data and displaying a user interface. A computing device or user device can send or receive signals, such as via a wired or wireless network, or can process or store signals, such as in memory as physical memory states. A device or user device as used in the present disclosure may include: a desktop computer; a mobile computer (e.g., a tablet computer, a laptop computer, or a notebook computer); a smartphone; a wearable computing device (e.g., smart watch); or the like, consistent with the computing device shown in FIG. 11.

As used herein, a “machine-learning model” generally encompasses instructions, data, and/or a model configured to receive input, and apply one or more of a weight, bias, classification, or analysis on the input to generate an output. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as k-nearest neighbors, linear regression, logistical regression, random forest, gradient boosted machine (GBM), support-vector machine, deep learning, text classifiers, image recognition classifiers, You Only Look Once (YOLO), a deep neural network, and/or any other suitable machine-learning technique that solves problems in the field of Natural Language Processing (NLP). Supervised, semi-supervised, and/or unsupervised training may be employed. For example, supervised learning may include providing training data and labels corresponding to the training data, e.g., as ground truth. Unsupervised approaches may include clustering, classification, principal component analysis (PCA) or the like. K-means clustering or K-Nearest Neighbors may also be used, which may be supervised or unsupervised. Combinations of K-Nearest Neighbors and an unsupervised cluster technique may also be used. Other models for detecting objects in contents/files, such as documents, images, pictures, drawings, and media files may be used as well. Any suitable type of training may be used, e.g., stochastic, gradient boosted, random seeded, recursive, epoch or batch-based, etc.

As used herein, “natural language processing (NLP)” generally encompasses techniques including, but not limited to, keyword search, finding relationships (e.g., synonyms, hypernyms, hyponyms, and meronyms), extracting information (e.g., keywords, key phrases, search terms), classifying, and determining positive/negative sentiment of articles.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

In certain non-limiting embodiments, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server, for example, can be a cloud-based server, a cloud-computing platform, or a virtual machine. Servers can vary widely in configuration or capabilities, but generally a server can include one or more central processing units and memory. A server can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For some non-limiting embodiments, a “network” should be understood to refer to a network that may couple devices so that communications can be exchanged, such as between a server and a user device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

Furthermore, devices or user devices, such as computing devices or other related electronic devices can be remotely coupled to a network, such as via a wired or wireless line or link, for example.

In certain non-limiting embodiments, a “wireless network” should be understood to couple user devices with a network. A wireless network can include virtually any type of wireless communication mechanism by which signals can be communicated between devices, between or within a network, or the like. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as user devices with varying degrees of mobility, for example.

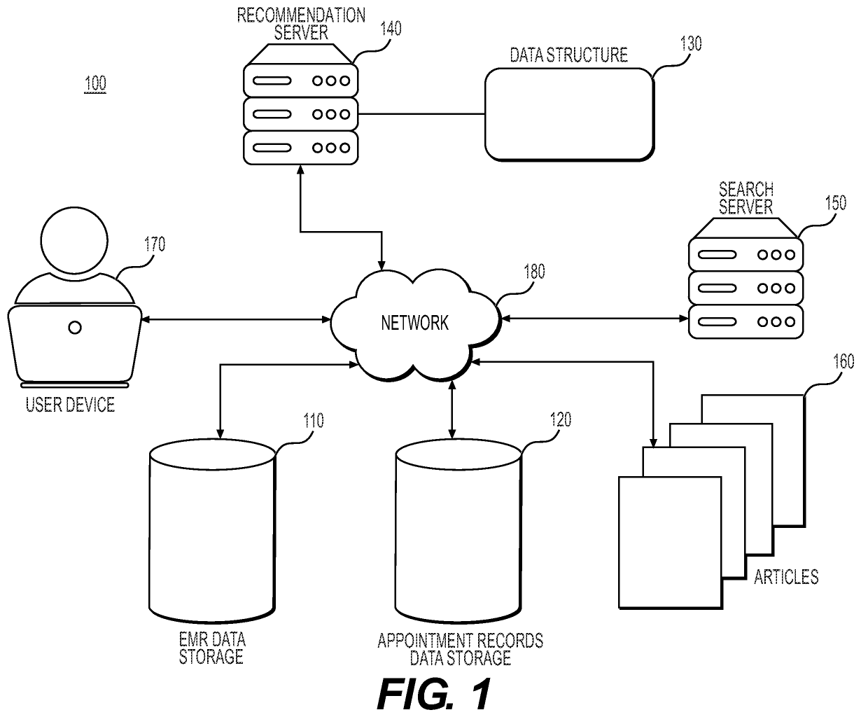

Referring now to the appended drawings, FIG. 1 shows an exemplary embodiment of a system 100 for recommending relevant articles. In general, system 100 may comprise EMR data storage 110, appointment records data storage 120, recommendation server 140, data structure 130, search server 150, article repository 160, user device 170, and network 180.

System 100 may be implemented on a network 180, allowing for the transmission or sharing of data between each of EMR data storage 110, appointment records data storage 120, recommendation server 140, data structure 130, search server 150, article repository 160, and user device 170 in a network environment. Some or all of the components illustrated in FIG. 1 may be implemented on a cloud platform.

EMR data storage 110 may be a data storage or a database storing EMRs associated with one or more patients. The EMRs of patients under the care of one or more clinicians may be stored in EMR data storage 110. In some examples, other health data associated with a patient such as a clinician's notes and/or recordings, other point-of-care documentation, and discharge documentation may be stored in EMR data storage 110.

Appointment records data storage 120 may be a data storage or a database, and may contain appointment records associated with one or more patients. The appointment records of patients under the care of one or more clinicians may be stored in appointment records data storage 120. Data associated with appointment records may be stored in appointment records data storage 120 upon the scheduling of a new appointment. In some examples, when a new appointment is scheduled for a patient, a record may be generated and may be transmitted to appointment records data storage 120 for storage. The record generated upon scheduling a new patient appointment may contain new appointment scheduling data. In some embodiments, EMR data storage 110 and/or appointment records data storage 120 may be a part of recommendation server 140.

Data structure 130 may be generated by recommendation server 140 and may be used to store historical patient data, such as data associated with EMR records. Data from the appointment records data storage 120 as well as the article repository 160 may also be added to and/or stored in the data structure 130. For instance, the data associated with EMR records, appointment records, and articles may be stored in the data structure 130 in a structured or organized manner. In a preferred embodiment, data structure 130 may be a graph data structure.

Recommendation server 140 may process data received from one or more of EMR data storage 110, appointment records data storage 120, user device 170, and article repository 160. Data processed by recommendation server 140 may be added to and/or stored in data structure 130. Recommendation server 140 may determine an appointment subject associated with new appoint scheduling data received from appointment records data storage 120. Recommendation server 140 may use data structure 130 to determine a correlation between the new appointment scheduling data and historical patient data. Recommendation server 140 may also process and analyze data in the data structure 130 to generate query data. Recommendation server 140 may transmit query data to search server 150 to query one or more databases, such as article repository 160, for one or more relevant articles. Recommendation server 140 may determine a relevance score for one or more relevant articles received from search server 150 and transmit a recommendation for one or more relevant articles to user device 170.

Search server 150 may receive query data from recommendation server 140. Search server 150 may query one or more relevant articles from article repository 160. Article repository 160 may be any source that may store one or more articles, such as, e.g., a server (hosting one or more websites), a data storage, or a database. Although depicted in FIG. 1 as a single repository, article repository 160 may comprise a plurality of servers, data storages, and/or databases storing a plurality of articles. In certain embodiments, article repository 160 may be a part of search server 150.

User device 170 may be used by a user, e.g., a clinician. User device 170 may receive a recommendation for one or more relevant articles. User device 170 may also be configured to input various patient and appoint-related information, which may be used to recommend medically-relevant articles using the techniques disclosed herein. User device 170 may display the one or more relevant articles through various displays in a manner that is user friendly and easily accessible for the clinician. In some examples, a clinician may be prompted, e.g., by recommendation server 140 to input patient information into user device 170. User device 170 may also be used to customize the display of the recommended articles.

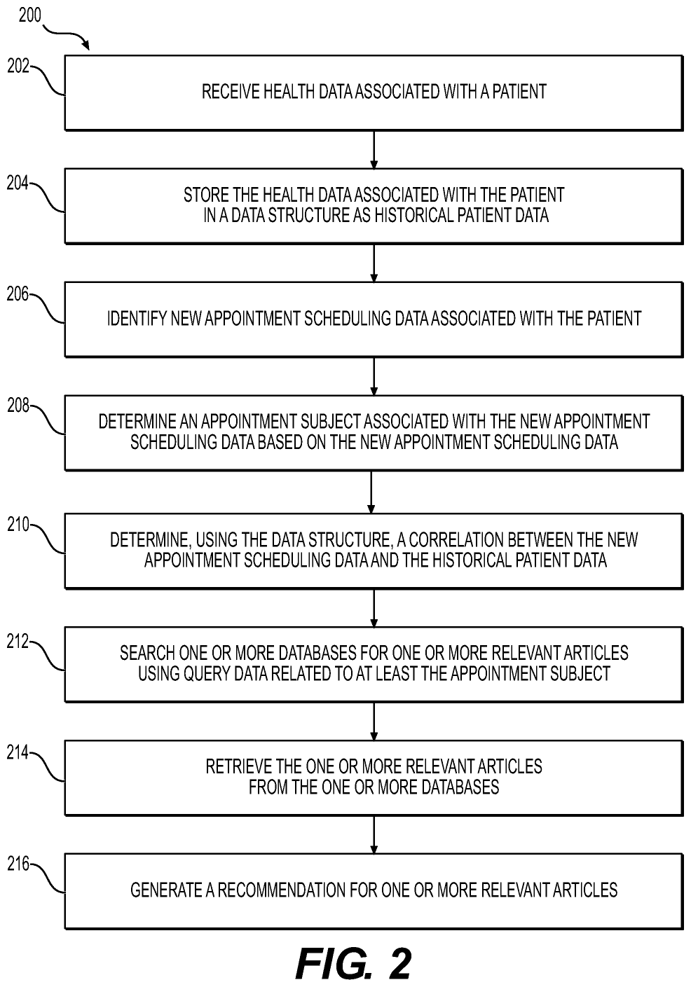

FIG. 2 shows an exemplary method 200 for recommending relevant articles. In step 202, health data associated with a patient may be received. For example, recommendation server 140 may receive health data associated with a patient via network 180 from EMR data storage 110. In some examples, health data associated with a patient may be received by recommendation server 140 from one or more sources, such as one or more data storages (e.g., EMR data storage 110 and appointment records data storage 120) and one or more user devices (e.g., user device 170). Health data associated with a patient may include electronic medical records (EMRs), appointment records, medication records, clinician's notes and/or recordings, and any other information relevant to a patient visit (e.g., other point-of-care documentation and discharge documentation).

Exemplary health data associated with a patient as provided by an EMR, appointment record, medication record, and/or other relevant patient information received in step 202 may include a clinical profile of the patient such as diseases, diagnosis, allergies, medications, procedures, significant medical events (e.g. heart attack, stroke, head trauma, transplant), disease stage, lab values, test results, patient self-reported clinical, behavioral and psycho-social data, a patient's demographics, medical history, family history, referral records, an appointment profile such as health care provider (e.g., treating clinician), date, day, time, location, duration of visit, and/or a decision which includes medication change, psycho-therapy, dose change, lifestyle modification (diet, exercise, weight loss), recommendation for lab test(s), and the like, and may also include the reasoning behind the clinician's decision(s).

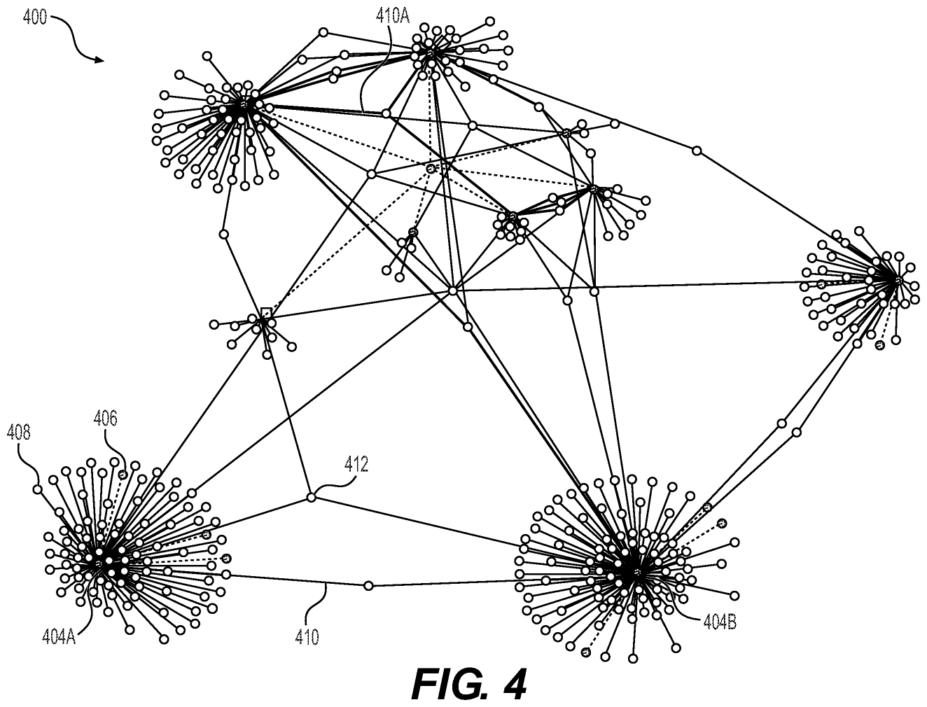

In step 204, recommendation server 140 may store the health data associated with the patient in a data structure as historical patient data. The historical patient data may include any data associated with a past visit of the patient. Any suitable data structure may be used to store the health data associated with the patient as historical patient data. For example, the data structure may be a graph data structure. An exemplary graph data structure may be a weighted graph as depicted in FIG. 4. The data structure (e.g. graph data structure) may be used to link or connect multiple patient visits based on similarity as discussed with respect to step 210 below.

In step 206, recommendation server 140 may identify new appointment scheduling data associated with the patient. In order to identify new appointment scheduling data for a patient, step 206 may include continuously, or periodically monitoring for each time a new appointment is scheduled for the patient. For example, an application may be used to monitor calendar data associated with a clinician to detect an upcoming patient appointment. The application may be used to detect a calendar notification generated each time a new appointment is scheduled. In some examples, a data storage storing appointment scheduling data may be accessed to retrieve data related to appointments recently scheduled to identify new appointment scheduling data. For example, an appointment records data storage 120 may be searched, such as by scanning or querying, for new appointment scheduling data.

The new appointment scheduling data identified in step 206 may include any information associated with the creation of a new appointment for the patient. For example, new appointment scheduling data may include the reason for the visit. Common reasons for scheduling a new appointment (i.e. reason for visit), may include cold/flu symptoms (e.g., sore throat, cough, nasal congestion), X-ray, joint pain, broken bones, generalized illness, infection (e.g., viral or bacterial), physical exam, shortness of breath, headache, abdominal pain, serious medical conditions (e.g., heart attack/disease, stroke, cancer, diabetes) maintenance, and follow-up. New appointment scheduling data may also include patient identification information, such as patient name and contact information, as well as date/time of appointment and clinician identification information (e.g., name of clinician and location). The new appointment scheduling data identified in step 206 may be generated as soon as a new appointment is scheduled. In cases where a patient is a walk-in and does not have a previously scheduled appointment, the new appointment scheduling data may be generated while the patient waits to see a clinician. The new appointment scheduling data may be identified in step 206 upon generation. In some examples, the new appointment scheduling data identified for the patient may be added to the data structure (e.g. graph data structure) storing the historical patient data from step 204.

In step 208, recommendation server 140 may determine an appointment subject associated with the new appointment scheduling data. In step 208, an appointment subject may be determined based on the new appointment scheduling data. For example, once the new appointment scheduling data is identified in step 206, the new appointment scheduling may be analyzed to determine or assign an appointment subject. An appointment subject may serve as a classification, which defines a context or subject of a visit. An appointment subject may be broken down into categories (e.g., classifications). Exemplary classifications may include, but are not limited to, side effects of a medication, new patient concern, allergies, progression of health history or health condition, genetic predisposition, post-operative follow-up, and post-operative complications. In some examples, the new appointment scheduling data for a patient may be analyzed with the historical patient data for the patient stored in the data structure in step 204. For example, when the new appointment scheduling data is identified and added to the data structure in step 206, the new appointment scheduling data may be analyzed and compared with the historical patient data (e.g., EMRs, appointment records, and other previous health data associated with the patient).

Analyzing the new appointment scheduling data with the historical patient data may help to define and/or provide context for the upcoming or new patient appointment associated with the new appointment scheduling data. A reason for the patient visit included with the new appointment scheduling data may be analyzed with the historical patient from the data structure to determine the appointment subject. Analyzing a reason for the patient visit with the historical patient data may categorize a subject of the appointment (i.e. an appointment subject) as medications and their side effects, new patient concern, allergies, genetic predisposition, post-operative follow-up, post-operative complication, progression of health history/condition, or the like. For example, if the reason for an upcoming patient visit is due to blurred vision, an analysis of the new appointment scheduling data with the historical patient data in the data structure may determine an appointment subject as a post-operative complication when the patient's EMR includes a recent cataract surgery. In another example, if the reason for an upcoming patient visit is attributed to arthritis, an analysis of the new appointment scheduling data with the historical patient data in the data structure may determine an appointment subject as a progression of a previous health condition and/or genetic predisposition, when the patient's EMR includes a previous joint surgery and a family history of arthritis. In yet another example, if the reason for an upcoming patient visit is a foot injury, an analysis of the new appointment scheduling data with the historical patient data in the data structure may determine a first appointment subject as a new patient concern and a second appointment subject as a post-operative follow-up, when the patient's historical data contains no previous record of a foot injury, but their EMR includes a recent (unrelated) appendectomy. In this case, a clinician may address both appointment subjects in the same visit.

Determining an appointment subject associated with a new appointment based on the new appointment scheduling data, such as by processing or analyzing the new appointment scheduling data with the historical patient data, may be performed using natural language processing (NLP) techniques. For example, natural language processing may be applied to the new appointment scheduling data or a combination of the new appointment scheduling data and the historical patient data in the data structure to determine an appointment subject. Known natural language processing techniques may be performed on the new appointment scheduling data to classify or categorize a reason for the new appointment, which may be considered to be the appointment subject. Alternatively, in some examples, the new appointment scheduling data or a combination of the new appointment scheduling data and the historical patient data may be analyzed by another technique such as, e.g., machine learning, statistical algorithms, or combinations thereof to determine an appointment subject.

In step 210, recommendation server 140 may determine a correlation between the new appointment scheduling data and the historical patient data using the data structure. For example, the new appointment scheduling data may be compared with the historical patient data in the data structure to determine the presence of any relationships and/or similarities between the new appointment and one or more previous appointments, which were previously stored in the data structure. In order to determine a correlation between the new appointment scheduling data and the historical patient data, a similarity scan may be performed on the new appointment scheduling data and the historical patient data within the data structure. The new appointment scheduling data may be scanned or analyzed against the historical patient data to determine a correlation or similarity between the appointment subject (e.g., reason) of the new appointment and historical patient data associated with one or more previous appointments.

Comparing the new appointment scheduling data with the historical patient data using the data structure may aid in classifying the reason for the new appointment and thus determining the appointment subject. For example, determining a correlation between a reason for an upcoming visit from the new appointment scheduling data and the historical patient data, may indicate that the reason for the new appointment is a result of the progression of a health condition previously reflected in the historical patient data. As such, in some examples, steps 208 and 210 may be performed concurrently. In other words, an appointment subject associated with the new appointment scheduling data discussed in step 208 and a correlation between the new appointment scheduling data and the historical patient data discussed in step 210 may be determined substantially simultaneously or in parallel using the data structure. Natural language processing techniques may also be performed to determine a correlation between the new scheduling data and the historical patient data in step 210.

In some examples, step 210 may also include determining a correlation between the appointment subject determined in step 208 and the historical patient data using the data structure. In these examples, the appointment subject determined in step 208 may be compared, such as by performing a similarity scan, with a specific subset of the historical patient data using the data structure. For example, the appointment subject associated with the new appointment scheduling data may be compared with one or more appointment subjects for one or more previous appointments stored in the data structure. One advantage in determining a correlation between the appointment subject associated with new appointment scheduling data and previous appointment subjects stored in the data structure as historical patient data, includes the ability to identify or detect multiple occurrences of the same appointment subject. An exact match between the appointment subject determined in step 208 and a previous appointment subject stored in the data structure as historical patient data may correspond to the identification of multiple occurrences of an appointment subject. In at least one example, when multiple occurrences of an appointment subject are identified, a display may be generated to display the multiple occurrences to a user (e.g., clinician). In addition to bolstering a search query for relevant articles, as discussed below, the identification of multiple occurrences of an appointment subject may also allow a clinician to review the outcomes for previous care plans associated with the previous appointment subject.

The correlation(s) determined in step 210 (e.g., between the new appointment scheduling data and the historical patient data) may be representative of similarities between the data. In some examples, values (e.g., computed values) may be used to represent the similarities. For example, a similarity score may be determined based on the correlation between the new appointment scheduling data and the historical patient data. In some examples, a similarity score may also be determined based on the correlation between the appointment subject associated with new appointment scheduling data and the historical patient data.

Determining a correlation between the new appointment scheduling data and the historical patient data using the data structure may include a similarity analysis. A similarity analysis, which may include a similarity scan, may be performed by natural language processing in order to determine a similarity score by comparing the new appointment scheduling data with the historical patient data. The similarity score may indicate a degree of correlation. A high correlation (e.g., high score) may indicate a high degree of similarity between the new appointment scheduling data and the patient historical data, which includes data associated with one or more previous appointments. In aspects of the present disclosure, similarity may be determined based on a cosine similarity. However, any other similarity metric, such as Jaccard similarity, Euclidean distance, or clustering, may be used to determine similarity.

In a preferred embodiment, the data structure used to store the historical patient data and to which the new appointment scheduling data may be added, may be a graph data structure. For example, relationships between existing historical patient data (e.g., relationships between previous patient appointments) as well as between the new appointment scheduling data and the historical patient data may be presented by a graph data structure, such as a weighted graph. The similarity scores determined in step 210 may be saved in the graph data structure. In some embodiments, a weighted graph may include nodes that represent each patient appointment and edges that connect the nodes representing patient appointments. Similarity between the connected nodes may be represented by the weight or length of the edges. A high correlation determined in step 210 may be represented by an edge connecting the new appointment with a previous appointment that is more weighted or of shorter length. An exemplary weighted graph according to the present disclosure, is described below with respect to FIG. 4. Other data structures suitable for representing relationships between various data points/nodes may also be used according to the present disclosure.

In some examples, although not depicted in the flowchart, an alert may be generated when there is no correlation between the new appointment scheduling data and the historical patient data. After step 210, if no correlation (e.g., no relationship or similarity) is determined using the data structure, an alert may be generated for the user (e.g., clinician). In certain cases, the reason for an upcoming patient appointment may be determined to be new (e.g., appointment subject is new) and may seem unrelated to any prior appointments or medical records. In other words, no correlation between the new appointment scheduling data and the historical patient data was determined. The generation of an alert may prompt the clinician to determine if the appointment subject associated with the new appointment scheduling data is an independent occurrence or a related progression of a previous appointment. For example, the clinician may determine that a patient's current joint pain is a progression of a joint surgery conducted years ago. In other examples, an alert may also be generated after step 210, when comorbidities are identified. For example, a correlation determined between the new appointment scheduling data and the historical patient data may demonstrate that a comorbidity may exist between the reason for the new patient appointment and a past diagnosis or disease.

In step 212, one or more databases or data storages may be searched for one or more relevant articles using query data related to at least the appointment subject associated with the new appointment scheduling data. Existing search engines may be used to search one or more medical literature databases, such as MEDLINE and EMBASE, for one or more relevant articles. For example, recommendation server 140 may generate and transmit query data to search server 150 in order to search databases or data storages associated with the article repository 160. In some embodiments, recommendation server 140 may have direct access to databases or data storages storing a plurality of articles and may query these databases or data storages using the query data, without transmitting the query data to another server that is configured to perform the search. The query data used to search or query the database(s) or data storage(s) may at least be based on the appointment subject associated with the new appointment scheduling data, which may encompass the reason for the patient visit. As such, known techniques for extracting keywords and/or key phrases may be used to generate key phrases associated with the appointment subject, including the reason for the visit.

The relationships (e.g., correlations or similarities) between the appointment subject associated with the new appointment scheduling data and the historical patient data may also be used to generate query data for the search. For example, when the new patient appointment is linked or connected to one or more previous patient appointments based on a determined correlation (e.g., similarity), the related historical patient data may also be used to construct the query data. The similarities between the appointment subject associated with the new appointment scheduling data for the new appointment and the historical patient data may be used to refine the search.

According to aspects of the present disclosure, one or more articles, or data identifying one or more articles, may also be stored in the data structure as historical patient data. The one or more articles may include one or more articles that were determined to be relevant to a previous patient visit. In some examples, each previous patient appointment represented as historical patient data in the data structure may also be associated with or linked to one or more articles relevant to the particular previous appointment. In some cases, the one or more relevant articles stored as historical patient data may also be shared between related past appointments, meaning the one or more relevant articles are connected or linked to both related past appointments. One or more articles that have been determined to be relevant to one or more past patient appointments that are similar to the new patient appointment may be analyzed or processed to extract keywords and/or key phrases. For example, one or more relevant articles from the historical patient data may be analyzed using NLP techniques and/or machine-learning models to identify keywords and/or key phrases. In at least one example, a Bidirectional Encoder Representation (BERT) model may be used, followed by using Rapid Automatic Keyword Extraction (RAKE) to analyze and extract keywords and/or key phrases from one or more relevant articles from the historical patient data.

After performing NLP techniques on the one or more relevant articles stored as historical patient data in the data structure, the output generated may also be stored in the data structure, e.g., graph data structure. The graph data structure may create relationships between the relevant articles from the historical patient data based on the keywords and/or key phrases determined by the natural language processing. The relationships determined by the data structure (e.g., graph data structure) may be combined with the keywords and/or key phrases determined by the NLP techniques to construct query data, e.g., one or more queries, which may be used to query one or more databases or data storages.

In some examples, keywords and/or key phrases may also be extracted from historical patient data determined to have a correlation (e.g., related historical patient data) with the new appointment scheduling data. For example, keywords from a patient's EMR may be used to query the one or more databases. In order to expand the search, related notes of an ICD-10 tree associated with an EMR may also be used.

Upon querying the one or more databases or data storages with the query data, one or more articles relevant to the new patient appointment may be determined by computing a relevance score. In some examples, a relevance score may be computed based on cosine similarity, which constructs a ratio of phrases in common pairwise between documents and yields a score between 0 and 1. The inputs for computing the relevance score may include key phrases identified by the natural language processing of the one or more related articles associated with the historical patient data, keywords associated with the new appointment scheduling data (e.g., reason for visit), related historical patient data, or any combination thereof.

For example, a relevance score may be determined based on a degree of relevance between the keywords from the articles returned from the one or more databases and the query data. The credibility of the sources of the articles may also be a factor in determining relevancy. Next, the determined relevance score may be compared to a predetermined threshold. An article may be determined to be relevant as related to the new patient appointment (e.g., appointment subject), or may be selected for recommendation to the user, if the determined relevance score exceeds or is equal to the predetermined threshold.

In step 214, the one or more relevant articles are retrieved from the one or more databases or data storages. For example, recommendation server 140 may retrieve the one or more relevant articles from article repository 160, directly or by way of search server 150. The one or more relevant articles, or data identifying the one or more relevant articles, may also be added to the data structure in association with the corresponding new appointment.

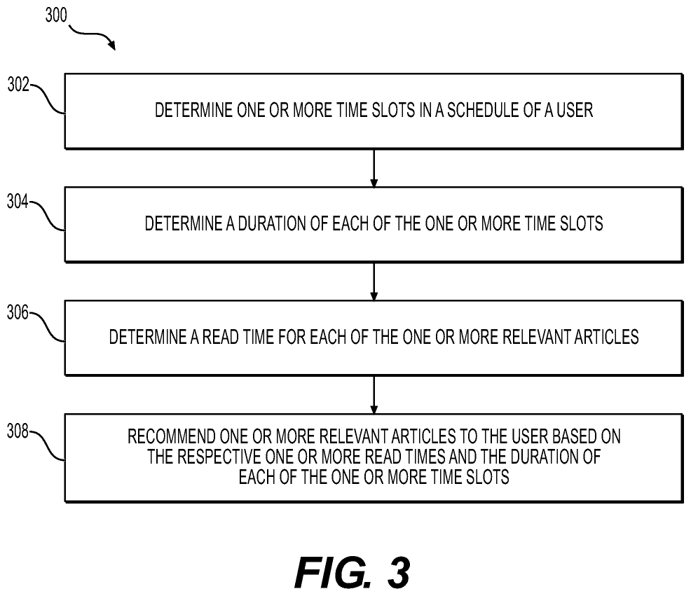

In step 216, recommendation server 140 may generate a recommendation for the one or more relevant articles. A variety of factors may contribute to the generation and subsequent display of the recommendation. For example, as discussed below with respect to method 300 of FIG. 3, in some embodiments, the one or more relevant articles may be recommended based on the amount of free time available in a clinician's schedule.

Although not depicted in the flowchart for method 200, additional steps may be performed. For example, the recommendation for the one or more relevant articles may be presented to a user (e.g., clinician) on a user device, such as user device 170. The recommendation for the one or more relevant articles may presented through at least one of a news feed, a calendar, or an electronic medical record (EMR) associated with the patient.

In some examples, the recommendation for the one or more relevant articles may be presented through all three options. Further, the one or more relevant articles may be presented in order of relevance to a patient. In some embodiments, the one or more relevant articles may be presented in order of relevance to a collection of patients with upcoming appointments. For example, when the one or more relevant articles are displayed through a personalized feed (e.g., a news feed) for upcoming patient appointments, a relevant article related to a patient with a serious health condition as determined by method 200, will be prioritized and appear at the top of the news feed. Relevant articles related to patients with less serious health conditions may appear later in the feed.

Further, regarding prioritization, in certain examples, the user may be prompted to enter one or more patients that have conditions that require a refresher. In these examples, conditions that may require a refresher, include conditions that a clinician may not have encountered or treated in years. When this is the case, system 100 may prioritize the one or more relevant articles related to those patients who require a refresher. For example, the one or more relevant articles related to patients who require a refresher may be displayed first in a personalized feed.

After step 216, all of the data associated with the new appointment for the patient may be stored in the data structure. For example, the appointment subject, data related to the correlation between the new appointment scheduling data and the historical patient data, and data related to the one or more relevant articles in the data structure may all be stored in data structure 130.

FIG. 3 shows an exemplary method 300 for recommending one or more relevant articles based on article read time and time slots in a schedule. In some embodiments of the present disclosure, the one or more relevant articles may be recommended based on the availability (e.g., free time) of a user (e.g., clinician) for reading the one or more relevant articles. As such, in certain embodiments, step 216 of method 200 may include performing the steps of method 300, in order to generate a recommendation for one or more relevant articles. Method 300 may be performed by recommendation server 140.

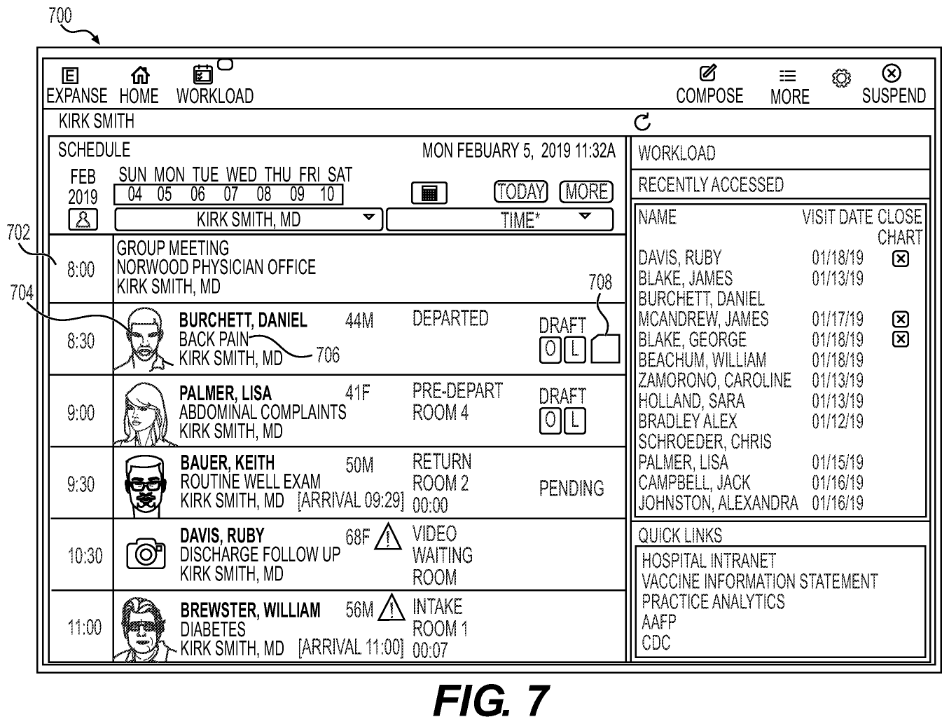

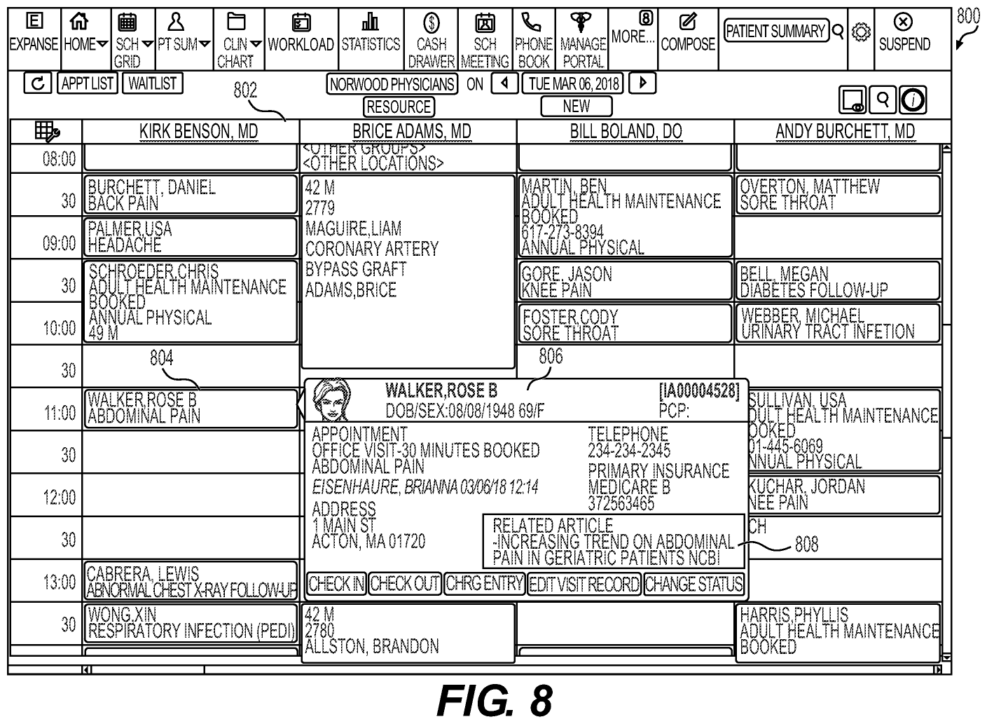

In step 302, one or more time slots in a schedule of a user may be determined. For example, a schedule (e.g., calendar) of a clinician which contains patient appointments may be accessed. Exemplary schedules which may be accessed are shown in FIGS. 7 and 8. Once a new appointment has been scheduled for a patient and added to the schedule of the user, the particular day for which the new appointment has been scheduled may be identified. The schedule of the user may be analyzed to identify one or more time slots. For example, the schedule of the user may be scanned to identify one or more time slots. In some examples, one or more time slots may constitute open, free, or otherwise available time slots within the user's schedule. In at least one example, once a new appointment for a patient has been scheduled, the clinician's calendar may be analyzed for all available time slots leading up to the patient's appointments. Available time slots prior to the patient's appointment may be determined for the same day as the appointment or in the days leading up to the appointment.

In step 304, a duration of each of the one or more time slots, which were determined to be available in step 302 may be determined. The duration of each of the one or more available time slots may be determined based on the start time and end time for each time slot. For example, an available time slot with a start time of 8:00 AM and an end time of 8:40 AM will have a duration of 40 minutes.

Step 306 includes accessing the one or more relevant articles retrieved in step 214 of method 200. In step 306, the one or more relevant articles may be analyzed to determine a read time for each of the one or more relevant articles. In some examples, natural language processing techniques and/or a machine learning model may be used to analyze each of the one or more relevant articles and determine an estimated reading time.

In step 308, one or more relevant articles may be recommended to the user based on the respective one or more read times determined in the step 306 and the duration of each of the one or more time slots (e.g., available time slots). The one or more relevant articles may only be recommended, if the estimated reading time fits within the duration of an available time slot of the user's schedule. For example, if the duration of an available time slot is 20 minutes, any relevant article that has an estimated reading time of 20 minutes or less may be recommended. When more than one relevant article is determined to have a read time (e.g., estimated reading time) that fits within a duration of an available time slot, the relevant article with the shortest read time may be recommended to the user first. The user may still have the option to view the other relevant articles with longer read times if desired. In some examples, the one or more relevant articles may be directly attached to an available time slot on the user's schedule based on the reading time relative to the duration of the time slot.

The recommendation for one or more relevant articles based on read time and time slots in a schedule may be conveyed in a variety of ways. In some examples, recommending the one or more relevant articles may include ordering the relevant articles based on the determined read time (e.g., from shortest read time to longest read time).

FIG. 4 depicts an exemplary graph data structure 400 representing multiple patient visits (i.e., appointments). Graph data structure 400 may be used as the data structure for storing historical patient data and determining a correlation between the new appointment scheduling data and the historical patient data in system 100 and method 200 as discussed above. Graph data structure 400 may be a weighted graph comprising a plurality of nodes and edges.

Graph data structure 400 may include data, such as new appointment scheduling data and historical patient data, for one or more patients. For example, a patient may be associated with a plurality of appointments 404, represented by nodes. Appointment 404A represents one appointment for the patient and appointment 404B represents a different appointment for the patient. There may be other nodes representing appointments for additional patients in graph data structure 400. In other words, graph data structure 400 may depict appointments associated with multiple patients. Graph data structure 400 may further depict the clinician(s) and one or more relevant articles associated with each patient appointment. For example, appointment 404A may be associated with clinician 406 and relevant article 408. In graph data structure 400, each circle without shading represents an article (e.g., articles 408, 412, etc.) that may be relevant to one or more appointments, the relevance to the one or more appointments being depicted by the connection or edge between the article and the one or more appointments. Each shaded circle situated in the middle of surrounding articles represents an appointment (e.g., appointments 404A, 404B, etc.), which may be associated with the articles and clinicians to which the appointment is connected. Each shaded circle that is connected to a corresponding appointment by a dotted line represents a clinician (e.g., clinician 406, etc.).

Graph data structure 400 may be used to link or connect patient appointments based on a determined correlation or similarity. For example, edge 410 connects appointment 404A and appointment 404B and may represent a relationship between appointment 404A and appointment 404B. For example, the appointment subject between appointment 404A and appointment 404B may be related. The edge weight may represent the degree of similarity. The edge weight may be represented by the length of the edge, or the distance between the nodes being connected by the edge. For example, the edge weight for edge 410A denotes a greater degree of similarity as compared to edge 410. Relevant article 412 represents an article that may be relevant to both appointment 404A and 404B.

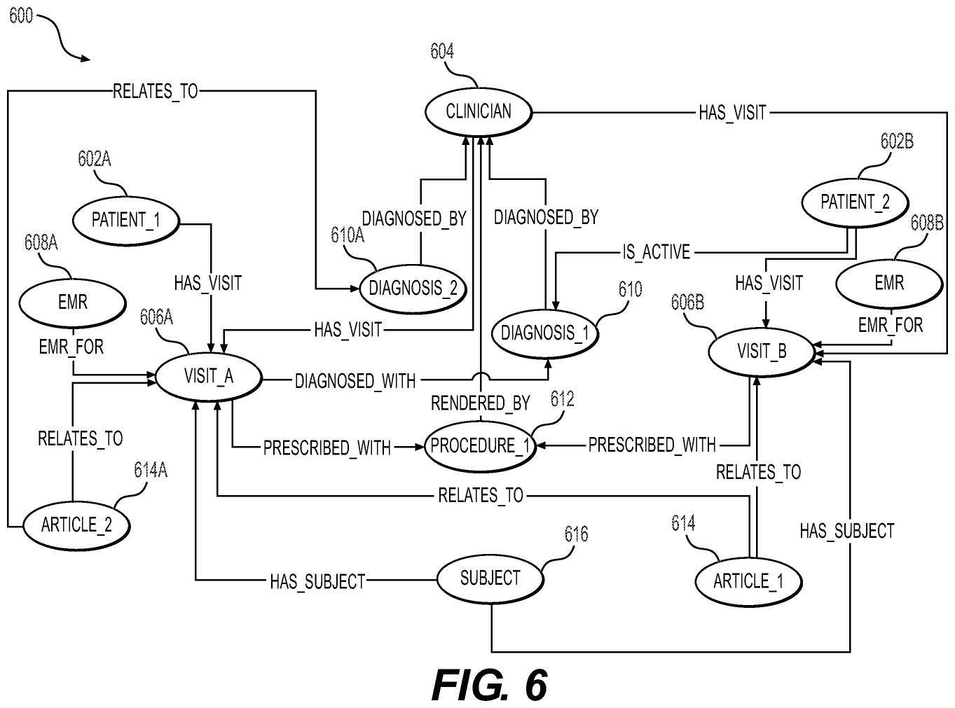

As discussed above, graph data structure 400 may be used for more than one patient with related data associated with their respective appointments. Such an example is described below in greater detail with respect to FIG. 6.

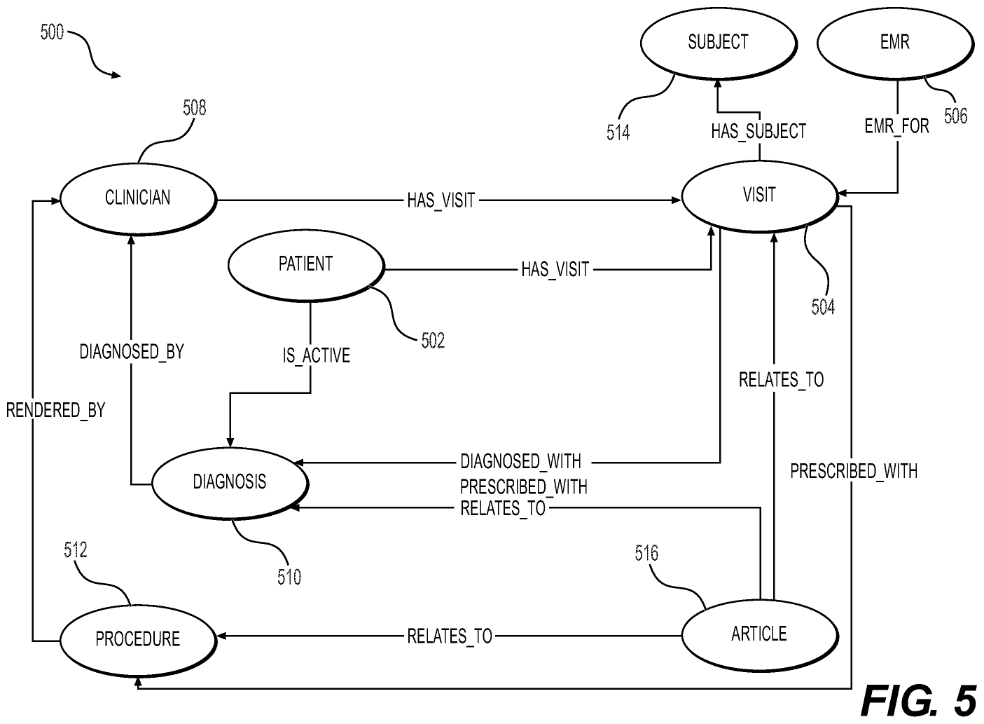

FIG. 5 shows an exemplary schema 500 of data in a graph data structure for a single patient. Schema 500 depicts in greater detail how data associated with a visit (i.e., appointment) for a patient may be organized or arranged in a graph data structure according to the present disclosure. For example, schema 500 depicts various nodes and connections/relationships therebetween that may be stored in a graph data structure for a patient. Schema 500 demonstrates that patient 502, clinician 508, and EMR 506 are associated with (e.g., connected to) visit 504 and that visit 504 has a subject 514 (e.g., appointment subject). Schema 500 also illustrates an article 516 that relates to (or is relevant to) visit 504, diagnosis 510, and procedure 512. Schema 500 further illustrates that diagnosis 510 and procedure 512 is diagnosed/rendered by clinician 508.

FIG. 6 shows an exemplary schema 600 of data in a graph data structure for multiple appointments. Schema 600 depicts in greater detail how data associated with two different visits (e.g., appointments) may be organized or arranged in a graph data structure according to the present disclosure. For example, schema 600 shows data associated with two visits (i.e., appointments), visit 606A and visit 606B. The visits 606A and 606B may be associated with a same patient, or with different patients. For example, patients 602A and 602B illustrated in the schema may be a same patient, or different patients. In a graph data structure according to the present disclosure, patient 602A may be connected to visit 606A and patient 602B may be connected to visit 606B. Both visits were attended to by the same (e.g., shared) clinician, clinician 604. Visits 606A and 606B also have the same subject, subject 616. Visits 606A and 606B are also connected through a shared diagnosis 610. Diagnosis 610A is unique to visit 606A and patient 602A. Patient 602B is still presenting symptoms of diagnosis 610. The clinician 604 rendered procedure 612 during or based on both visit 606A and visit 606B. Article 614 relates to both visits, while article 614A relates only to visit 606A and diagnosis 610A.

FIG. 7 shows an exemplary clinician calendar 700. Specifically, the calendar shown in FIG. 7 displays appointment entries for a selected date. Clinician calendar 700 displays an appointment time slot 702 for each time slot on a clinician's schedule, and a patient profile 704 for each patient scheduled within the respective time slot. Clinician calendar 700 also displays a visit reason 706 (e.g., back pain) for the appointment. At least one relevant article 708 as recommended in step 216 of method 200 may be displayed as a direct calendar attachment. A user may access the relevant article 708 by clicking on it.

FIG. 8 shows an exemplary clinician calendar 800. Clinician calendar 800 represents a calendar accessible to and representative of multiple clinicians, e.g., a physician practice group. Calendar 800 displays a column 802 for each clinician's schedule of patient appointments. A patient appointment 804 may be displayed as a calendar entry for a particular time slot. A popup notification 806 of a patient's profile may be displayed when the user selects or hovers over patient appointment 804. A relevant article 808 is attached to and contained within the popup notification of the patient's profile and includes a brief summary of the article. The user may access the full article by clicking on the article 808 attached to the popup notification.

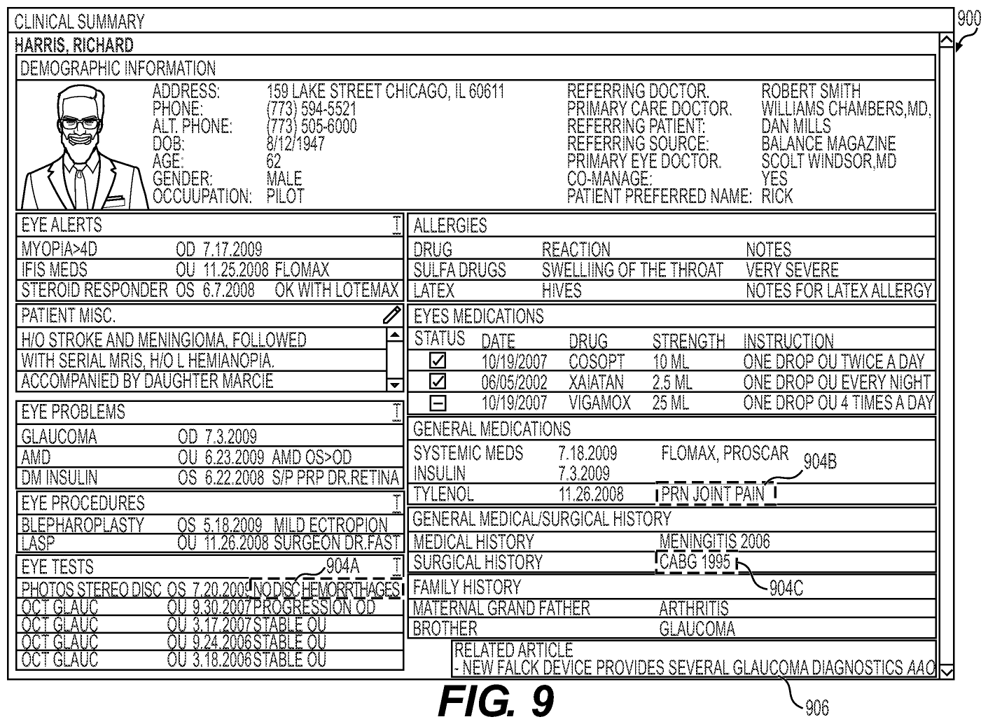

FIG. 9 shows an exemplary electronic medical record (EMR) 900. EMR 900 displays information for a patient commonly displayed on EMRs such as demographic information, allergies, medications, and medical history. In addition, relevant article 906 is attached to and contained within the EMR. The portions 904A, 904B, and 904C of the EMR that were used to recommend the relevant article 906 are highlighted. The highlighted portions may include keywords and/or phrases used to construct the search query or compare with other patient historical data.

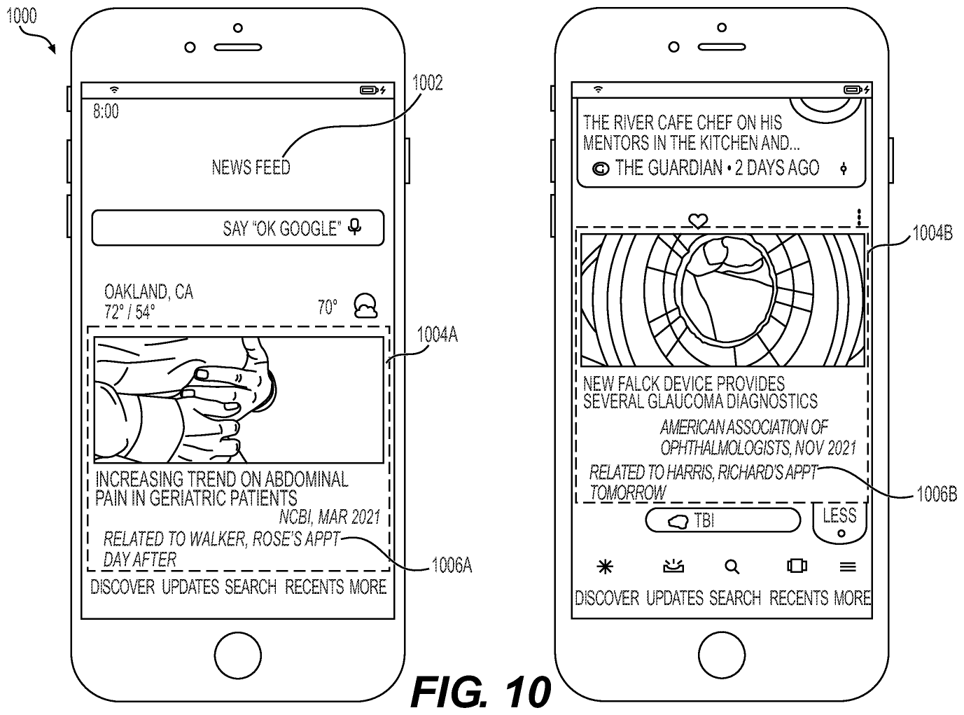

FIG. 10 shows an exemplary personalized feed display 1000. Personalized feed display 1000 may be generated on a user (e.g., clinician) device as a news feed 1002. Relevant articles 1004A and 10046 may be displayed in the clinician's news feed 1002. In addition to displaying descriptions for the articles including the title and the source, the news feed 1002 may display the contextual information 1006, such as 1006A and 1006A, for the relevant article. The contextual information 1006 may describe to which appointment(s) the article is relevant by displaying the appointment time, patient name, and any other information associated with the appointment.

Regarding the presentation and display of the one or more relevant articles recommended according to the present disclosure, the relevant articles may be displayed and presented to the user in various levels of summarization. For example, with respect to the exemplary personalized feed display 1000, in addition to displaying the title of the relevant articles, a short summary and/or abstract (if available) may be displayed. The level of summarization may be determined based on the clinician's availability (e.g., time available for reading the article, such as a free time between appointments) and duration of the availability at the time the article is recommended to the clinician. As demonstrated with the exemplary EMR 900, keywords may also be displayed. Another display option may also include displaying the “read time” determined in method 300 with the one or more relevant articles, so that the user may decide if they want to read the article now or at a later time.

FIG. 11 illustrates an implementation of a computer system that may execute techniques presented herein. The computer system 1100 can include a set of instructions that can be executed to cause the computer system 1100 to perform any one or more of the methods or computer based functions disclosed herein. The computer system 1100 may operate as a standalone device or may be connected, e.g., using a network, to other computer systems or peripheral devices.

In a networked deployment, the computer system 1100 may operate in the capacity of a server or as a client user computer in a server-client user network environment, or as a peer computer system in a peer-to-peer (or distributed) network environment. The computer system 1100 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 1100 can be implemented using electronic devices that provide voice, video, or data communication. Further, while a single computer system 1100 is illustrated, the term “system” shall also be taken to include any collection of systems or sub-systems that individually or jointly execute a set, or multiple sets, of instructions to perform one or more computer functions.

As illustrated in FIG. 11, the computer system 1100 may include a processor 1102, e.g., a central processing unit (CPU), a graphics processing unit (GPU), or both. The processor 1102 may be a component in a variety of systems. For example, the processor 1102 may be part of a standard personal computer or a workstation. The processor 1102 may be one or more general processors, digital signal processors, application specific integrated circuits, field programmable gate arrays, servers, networks, digital circuits, analog circuits, combinations thereof, or other now known or later developed devices for analyzing and processing data. The processor 1102 may implement a software program, such as code generated manually (i.e., programmed).

The computer system 1100 may include a memory 1104 that can communicate via a bus 1108. The memory 1104 may be a main memory, a static memory, or a dynamic memory. The memory 1104 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 1104 includes a cache or random-access memory for the processor 1102. In alternative implementations, the memory 1104 is separate from the processor 1102, such as a cache memory of a processor, the system memory, or other memory. The memory 1104 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 1104 is operable to store instructions executable by the processor 1102. The functions, acts or tasks illustrated in the figures or described herein may be performed by the programmed processor 1102 executing the instructions stored in the memory 1104. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 1100 may further include a display unit 1010, such as a liquid crystal display (LCD), an organic light emitting diode (OLED), a flat panel display, a solid-state display, a cathode ray tube (CRT), a projector, a printer or other now known or later developed display device for outputting determined information. The display 1110 may act as an interface for the user to see the functioning of the processor 1102, or specifically as an interface with the software stored in the memory 1104 or in the drive unit 1106.

Additionally or alternatively, the computer system 1100 may include an input device 1112 configured to allow a user to interact with any of the components of system 1100. The input device 1112 may be a number pad, a keyboard, or a cursor control device, such as a mouse, or a joystick, touch screen display, remote control, or any other device operative to interact with the computer system 1100.

The computer system 1100 may also or alternatively include a disk or optical drive unit 1106. The disk drive unit 1106 may include a computer-readable medium 1122 in which one or more sets of instructions 1124, e.g. software, can be embedded. Further, the instructions 1124 may embody one or more of the methods or logic as described herein. The instructions 1124 may reside completely or partially within the memory 1104 and/or within the processor 1102 during execution by the computer system 1100. The memory 1104 and the processor 1102 also may include computer-readable media as discussed above.

In some systems, a computer-readable medium 1122 includes instructions 1124 or receives and executes instructions 1124 responsive to a propagated signal so that a device connected to a network 1150 can communicate voice, video, audio, images, or any other data over the network 1150. Further, the instructions 1124 may be transmitted or received over the network 1150 via a communication port or interface 1120, and/or using a bus 1108. The communication port or interface 1120 may be a part of the processor 1102 or may be a separate component. The communication port 1120 may be created in software or may be a physical connection in hardware. The communication port 1120 may be configured to connect with a network 1150, external media, the display 1110, or any other components in system 1100, or combinations thereof. The connection with the network 1150 may be a physical connection, such as a wired Ethernet connection or may be established wirelessly as discussed below. Likewise, the additional connections with other components of the system 1100 may be physical connections or may be established wirelessly. The network 1150 may alternatively be directly connected to the bus 1108.

While the computer-readable medium 1122 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 1122 may be non-transitory, and may be tangible.

The computer-readable medium 1122 can include a solid-state memory such as a memory card or other package that houses one or more non-volatile read-only memories. The computer-readable medium 1122 can be a random-access memory or other volatile re-writable memory. Additionally or alternatively, the computer-readable medium 1122 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 1100 may be connected to one or more networks 1150. The network 1150 may define one or more networks including wired or wireless networks. The wireless network may be a cellular telephone network, an 802.11, 802.16, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 1050 may include wide area networks (WAN), such as the Internet, local area networks (LAN), campus area networks, metropolitan area networks, a direct connection such as through a Universal Serial Bus (USB) port, or any other networks that may allow for data communication. The network 1150 may be configured to couple one computing device to another computing device to enable communication of data between the devices. The network 1150 may generally be enabled to employ any form of machine-readable media for communicating information from one device to another. The network 1150 may include communication methods by which information may travel between computing devices. The network 1150 may be divided into sub-networks. The sub-networks may allow access to all of the other components connected thereto or the sub-networks may restrict access between the components. The network 1150 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

We claim:

1. A computer-implemented method for recommending medically-relevant articles, the method comprising:
receiving, by a processor, health data associated with a patient;
storing, by the processor, the health data associated with the patient in a data structure as historical patient data;
identifying, by the processor, new appointment scheduling data associated with the patient;
determining, by the processor, an appointment subject associated with the new appointment scheduling data based on the new appointment scheduling data;
determining, by the processor and using the data structure, a correlation between the new appointment scheduling data and the historical patient data based on the new appointment scheduling data and the historical patient data;
searching, by the processor, one or more databases for one or more relevant articles using query data related to at least the appointment subject;
retrieving, by the processor, the one or more relevant articles from the one or more databases; and
generating, by the processor, a recommendation for the one or more relevant articles.

2. The computer-implemented method of claim 1, further comprising:
presenting the recommendation for the one or more relevant articles to a user on a user device through at least one of a news feed, a calendar, or an electronic medical record (EMR) associated with the patient.

3. The computer-implemented method of claim 1, wherein the one or more relevant articles are presented to a user in different levels of summarization.

4. The computer-implemented method of claim 1, wherein the data structure is a graph data structure.

5. The computer-implemented method of claim 4, wherein the graph data structure is a weighted graph, the weighted graph comprising a plurality of nodes representing each appointment of the patient, wherein each node is associated with an appointment subject, historical patient data, and one or more relevant articles, wherein the nodes are connected based on the respective appointment subjects, and wherein edges between the connected nodes represent similarities between the connected nodes.

6. The computer-implemented method of claim 1, wherein the health data associated with a patient comprises one or more electronic medical records and one or more appointment records.

7. The computer-implemented method of claim 1, wherein the new appointment scheduling data comprises a reason an appointment is scheduled for the patient.

8. The computer-implemented method of claim 1, wherein determining an appointment subject associated with the new appointment scheduling data comprises performing natural language processing on the new appointment scheduling data to determine the appointment subject.

9. The computer-implemented method of claim 8, wherein the appointment subject comprises one or more of a side effect of a medication, a new patient concern, an allergy, or a progression of a health condition.

10. The computer-implemented method of claim 1, wherein determining a correlation between the new appointment scheduling data and the historical patient data comprises searching the historical patient data stored in the data structure for data related to the new appointment scheduling data, wherein the correlation is represented by a similarity score.

11. The computer-implemented method of claim 1, wherein searching one or more databases for one or more relevant articles comprises:
computing a relevance score for each of a plurality of articles stored in the one or more databases; and
selecting one or more articles with a relevance score above a threshold, wherein the relevance score of each of the plurality of articles is relative to the appointment subject and/or the correlation between the new appointment scheduling data and the historical patient data and/or one or more articles associated with related historical patient data.

12. The computer-implemented method of claim 1, wherein searching one or more databases for one or more relevant articles comprises:
querying the one or more databases with the query data related to at least the appointment subject;
wherein the query data used is generated based on key phrases extracted from one or more articles associated with related historical patient data or by combining key phrases extracted from the new appointment scheduling data, related historical patient data, and one or more articles associated with the related historical patient data.

13. The computer-implemented method of claim 1, further comprising:
generating an alert when there is no correlation between the new appointment scheduling data and the historical patient data; or
generating an alert when a comorbidity is detected when determining a correlation between the new appointment scheduling data and the historical patient data.

14. The computer-implemented method of claim 1, further comprising:
storing the appointment subject, data related to the correlation between the new appointment scheduling data and the historical patient data, and data related to the one or more relevant articles in the data structure.

15. The computer-implemented method of claim 2, wherein when the one or more relevant articles are presented in an EMR associated with the patient, the one or more relevant articles are attached to the EMR and one or more portions of the EMR that are relevant to the appointment subject are highlighted.

16. The computer-implemented method of claim 2, further comprising:
prompting the user to input one or more patients that have conditions that require a refresher; and
prioritizing one or more relevant articles related to the one or more patients in the news feed.

17. The computer-implemented method of claim 1, wherein generating a recommendation for the one or more relevant articles comprises:
determining, by the processor, one or more time slots in a schedule of a user;
determining, by the processor, a duration of each of the one or more time slots;
determining, by the processor, a read time for each of the one or more relevant articles; and
recommending, by the processor, one or more relevant articles to the user based on the respective one or more read times and the duration of each of the one or more time slots.

18. The computer-implemented method of claim 1, further comprising:
displaying, by the processor, multiple occurrences of an appointment subject after analyzing the new appointment scheduling data with the historical patient data.

19. A system for recommending medically-relevant articles, the system comprising:
a memory having processor-readable instructions stored therein; and
a processor configured to access the memory and execute the processor-readable instructions to perform operations comprising:
receiving health data associated with a patient;
storing the health data associated with the patient in a data structure as historical patient data;
identifying new appointment scheduling data associated with the patient;
determining an appointment subject associated with the new appointment scheduling data based on the new appointment scheduling data;
determining, by using the data structure, a correlation between the new appointment scheduling data and the historical patient data based on the new appointment scheduling data and the historical patient data;
searching one or more databases for one or more relevant articles using query data related to at least the appointment subject;
retrieving the one or more relevant articles from the one or more databases;
generating a recommendation for the one or more relevant articles; and
presenting the recommendation for the one or more relevant articles to a user through at least one of a news feed, a calendar, or an electronic medical record (EMR) associated with the patient.

20. A non-transitory computer-readable medium storing a set of instructions that, when executed by a processor, perform operations for recommending medically-relevant articles, the operations comprising:
receiving health data associated with a patient;
storing the health data associated with the patient in a data structure as historical patient data;
identifying new appointment scheduling data associated with the patient;
determining an appointment subject associated with the new appointment scheduling data based on the new appointment scheduling data;
determining, by using the data structure, a correlation between the new appointment scheduling data and the historical patient data based on the new appointment scheduling data and the historical patient data;
searching one or more databases for one or more relevant articles using query data related to at least the appointment subject;
retrieving the one or more relevant articles from the one or more databases;
generating a recommendation for the one or more relevant articles; and
presenting the recommendation for the one or more relevant articles to a user through at least one of a news feed, a calendar, or an electronic medical record (EMR) associated with the patient.
